(19) United States
(12) Patent Application Publication
(10) Pub. No.: US 2007/0250082 A1
(43) Pub. Date: Oct. 25, 2007

(54) Anastomosis Device and Method

(76) Inventor: Hassan Abou Kansoul, Norsborg (SE)

Correspondence Address: DRINKER BIDDLE & REATH (DC), 1500 K STREET, N.W., SUITE 1100, WASHINGTON, DC, 20005-1209, US

(21) Appl. No.: 11/632,909

(22) PCT Filed: Jul. 7, 2005
(86) PCT No.: PCT/SE05/01131

§ 371 Date: Jan. 19, 2007

(30) Foreign Application Priority Data
Jul. 22, 2004 (SE) 0401917-0

(51) Int. Cl. A61B 17/08

(52) U.S. Cl. 606/153

(57) ABSTRACT

The invention relates to an anastomosis device for anastomosing the end of two vessel parts together. It includes two attachment units attachable to a respective end of the vessel parts. The units are connectable to each other. Each unit has a through-hole defining a central axis. According to the invention the device has at least two connection means. Each connection means includes an axially extending member having a free end, which member is provided on any of the attachment units and a receiving opening provided on the other of the attachment units. The receiving opening is arranged to axially receive the free end of the member. The member is arranged to snap into a locking position in the receiving opening in which the member is axially locked. The invention also relates to a method for anatomising of ends of two vessel parts together and to a use of the device.

### FIELD OF INVENTION

The present invention in a first aspect relates to an anastomosis device for anastomosing the ends of two vessels parts or other tubular organs together, the device including first and second attachment units attachable to a respective end of the vessel parts and connectable to each other, each attachment unit having a through-hole defining a central axis of the attachment unit. Normally, but not necessarily the through-hole is circular.

In a second aspect the invention relates to a method for anastomosing of ends of two vessel parts together by attaching first and second attachment units to a respective end of the vessel parts and connecting the attachment units to each other, each attachment unit having a through-hole defining a central axis of the attachment unit.

In a third aspect the invention relates to a use of the invented device.

In this application definitions like axial, radial, tangential are related to said central axis.

### BACKGROUND OF THE INVENTION

Anastomosis is the surgical joining biological tissue to create an internal communication between them. Vascular surgery often involves creating an anastomosis between blood vessels to create or restore blood flow to essential tissues. At present, the majority of all vascular anastomosis procedures are performed by conventional hand suturing. Suturing an anastomosis is time consuming and difficult. It is important that each anastomosis provide a smooth and open flow path and the attachment must be leak free under relatively high pressures. A completely leak free anastomosis is not always achieved on the first try. Consequently, there is a frequent need to re-suture the anastomosis to close any leaks or remove any flow interruptions that are detected.

Attempts has been made to provide devices and methods that allow more reliable anastomosis and which require shorter operation time.

Examples of such devices and methods are disclosed in U.S. Pat. No. 3,258,012, U.S. Pat. No. 4,917,090 and U.S. Pat. No. 6,524,322.

U.S. Pat. No. 3,258,012 discloses a method of connecting blood vessels using a pair of needle discs having alternately placed projecting needles and receiving bores on an annular base member, and forceps having clamping jaws forming, when clamped together, an annular groove in which the discs may be positioned, comprising directing a first blood vessel through a bore of forceps and a needle disc positioned in the groove thereof and securing the outer walls of the blood vessel to the needles projecting therefrom, directing a second blood vessel through a second forceps through the bore thereof and the bore of a needle disc held thereby and anchoring the walls to the needles projecting therefrom, positioning the two of said forceps so that the needles of one disc align with the openings defined in the other, and pressing the forceps together to cause the needles of one disc to enter the bores of the other and to deflect the needles around the exterior walls of opposing discs to interlock said discs and said blood vessels.

U.S. Pat. No. 4,917,090 discloses fasteners for the joining of blood vessels or tubular organs. The fasteners are shaped in such a manner that they are retained in a novel clamping device until the joining is accomplished. The fasteners comprise at least two couplable rings having axially directed pins and intermediate holes distributed about their centers, and a shoulder portion formed along the periphery of at least one of the rings for fitting at least one of the rings into the clamping device.

U.S. Pat. No. 6,524,322 discloses an anastomotic device for joining vessels, in the first place joining one end of a graft vessel to a target vessel at an opening made in the wall thereof. The anastomotic device comprises a tubular body on which an outer flange, which comes into contact with the outside of the wall of the target vessel around the opening, and an inner flange, which comes into contact with the inside of the wall of the target vessel around the opening, are arranged. The inner flange is made up of a number of arms which are able to move from an extended position, located in the extension of the tubular body, under the influence of a pretension into a position extending in the lateral direction with respect to the tubular body, after the pretension has been released, in order to form the inner flange. In FIGS. 18-22 in particular end-to-end joining is disclosed.

In the prior art disclosures the connection of the two attachments units are either not secure or complicated or entails both these drawbacks.

The object of the present invention is to arrive to an anastomosis device in which these drawbacks are overcome and thus results in a device where it is easy to connect the attachment units to each other and simultaneously assures a reliable connection.

### DESCRIPTION OF THE INVENTION

This object has according to the first aspect of the invention been achieved in that an anastomosis device according to the preamble of claim 1 includes the specific features that the device is provided with at least two connection means, each connection means including an axially extending member having a free end, which member is provided on any of the attachment units and a receiving opening provided on the other of the attachment units, which receiving opening is arranged for axially receiving the free end of said member and which member is arranged to be able to snap into a locking position in said receiving opening in which position the member is axially locked.

By providing members that snap into receiving openings the connection can be done very easy and without any risk for default. When the members have snapped into the receiving openings the axially locking of the connection eliminates the risk that the two units loosen from each other. The units thus can be promptly interconnected assuring safe anastomosis.

According to a preferred embodiment of the invented device all of said members are provided on the first attachment unit and all of said receiving openings are provided on the seconds attachment units.

This simplifies the connection movement when the two units are moved towards each other due to the symmetry thus achieved. It is also more convenient if one of the units is entirely of the male-type and the other of female-type.

According to a further preferred embodiment each member has such a shape that it is flexible to or from the central axis of the through-hole of the first attachment unit.

The flexibility allows a simple and reliable way of obtaining the snap-action and locking position. By arranging the flexibility in the mentioned direction it is easy to manipulate the member when connecting the units to each other.

According to a further preferred embodiment each member has a tongue-like shape having its thinnest dimension in the radial direction.

Through such a shape of the member it is attained that flexibility in the direction to or from the central axis easily is established while maintaining a sufficient cross-sectional area of the member for taking up forces. This shape also facilitates the manipulation of the device when the units are to be connected to each other.

According to a further preferred embodiment the free end of each member is provided with at least one tangentially extending shoulder, and one wall of each receiving opening is provided with at least one tangentially extending shoulder.

By such shoulders the locking of the units to each other after the snapping in of the members assures that the parts are securely held together. The co-operating shoulders is a very uncomplicated way of obtaining this locking.

According to a further preferred embodiment there is a plurality of axially distributed shoulders on each member and/or in each receiving opening such that a saw-tooth-like profile is formed on each member and in each receiving opening.

To assure a safe and firm anastomosis, the surgeon has to achieve a proper tight anastomosis not to have necrosis of the tissue but provide a safe, connection between the vessels. By the serrated members and/or receiving openings the surgeon can control the tightness of the anastomosis. Thus, by varying the force when interconnecting the units, the surgeon can control the tightness of the anastomosis by controlling the distance on which the saw-teeth are over-lapping inside the receiving opening when the members are slipped in. In this way a safe, tight, leakage free, easy and quick anastomosis can be made.

According to a further preferred embodiment each attachment unit includes penetration means arranged to penetrate the walls of the related vessel thereby establishing attachment of the unit to the end of the vessel.

This way of attaching each attachment unit to its respective vessel end is time-effective and also allows a very secure attachment.

According to a further preferred embodiment the penetration means includes a plurality of axially directed projections, each projection having a sharp axially directed edge.

By providing axially directed projections with sharp edges the penetration is easily obtained and offers the possibility to fold the vessel ends around the projections to secure a strong attachment.

According to a further preferred embodiment each attachment unit is provided with a plurality of holes arranged to receive the edges of the axially directed projections of the other unit. Thereby the tightness of the connection of the two vessel parts is enhanced.

According to a further preferred embodiment each attachment unit is provided with a circumferentially extending rim of silicon arranged to receive the edges of the axially directed projections of the other unit. That is an alternative way of enhancing the tightness of the connection. This alternative has the advantage that the two units do not require to be precisely aligned in the circumferential direction.

According to a further preferred embodiment each projection has a substantially triangular shape and has an acute angle forming said sharp edge, or each projection is shaped as a needle.

Such shapes of the penetration means are advantageous for the penetration step as well as the folding step and facilitate the attachment.

According to a further preferred embodiment the projections are circumferentially distributed around the through-hole.

Thereby is assured that a complete connection is obtained around the entire vessel end and minimizes the risk for leakage.

According to a further preferred embodiment one of the attachment units is provided with a circumferentially extending rim on an axially directed surface thereof and the other attachment unit has a circumferentially extending groove on an axially directed surface thereof, the groove being arranged to receive the rim when the attachment units are connected to each other. This assures a leakage free connection of units.

According to a further preferred embodiment a main part of each attachment unit is a substantially rotational-symmetric body.

Since the vessels that are to be anastomosed to each other normally have circular cross-sections such a shape of the attachment unit optimises their adaptation.

According to a further preferred embodiment at least one of said attachement units includes a supply opening and at least one outlet opening in communication with the supply opening, the supply opening being arranged to be connectable to a conduct, and each outlet opening being located such that it faces the interior of the vessel when the attachement unit is attached to the vessel.

Sometimes there are risks of complications after surgical interventions on vessels. One of the most common and serious complications is thrombosis. If an anastomosis is not well done, it can lead also to hemorrhagic complications. Frequently vessel stenosis can appear after anastomosis.

Often drugs which can be used to prevent trombosis are administrated generally, but in that way they have general effects. These effects are benefice at the operation sites, but can give complications in other parts of the body. The tendencies are to somehow localize these effects to the site of interest.

By providing a supply opening in one of the attachment units a drug can be delivered locally to the area where the above described problem occur. Thereby the risk for thrombosis and other complications can be radically reduced and simultaneously it is avoided that other parts of the person's body is affected by the drug.

According to a further preferred embodiment at least one of said axially directed projections includes said at least one outlet opening located at or adjacent to the sharp edge of the projection.

Since the edges of the axially directed projections will be located inside the vessel when the unit is attached thereto this offers an advantageous way of distributing the drug to the anastomosis region.

According to a further preferred embodiment each axially directed projection that includes one of said at least one outlet opening is slightly inclined radially inwards in direction towards its edge.

By inclining these projections inwards the drug will be directed effectively to the area where it is most needed.

The above described preferred embodiments of the invented device are specified in the claims dependent from claim 1.

According to the second aspect of the invention the object of the invention has been achieved in that a method of the kind specified in the preamble of claim 19 includes the specific measures that the attachment units are connected to each other by at least two connection means, each connection means including an axially extending member provided on any of the attachment units and a receiving opening provided on the other of the attachment units, whereby the member is axially inserted into said receiving opening and snaps into the receiving opening such that the member becomes axially locked in the receiving opening.

By the invented method and the preferred embodiments thereof advantages are gained corresponding to the advantages of the invented device and the preferred embodiments thereof which have been described above.

### BRIEF DESCRIPTION OF THE DRAWINGS

FIG. 1 is a perspective view of a first unit of a device according to the invention.

FIG. 2 is a perspective view of a second unit of a device according to the invention.

FIG. 3 is a perspective view of the units of FIGS. 1 and 2 being interconnected.

FIG. 4 is an enlarged section through details of the invention.

FIG. 5 is an enlarged section trough corresponding details of an alternative example of the invention.

FIG. 6 in a perspective view illustrates the attachment of the unit of FIG. 1 to a vessel end.

FIG. 7 in a perspective view illustrates the attachment of the unit of FIG. 2 to a vessel end.

FIG. 8 in a side view illustrates the device according to the invention connecting two vessels to each other.

FIG. 9 is a perspective view of an alternative example of the unit shown in FIG. 1.

FIG. 10 is a perspective view of a still alternative example of the unit shown in FIG. 1.

FIG. 11 is a perspective view of an alternative example of the unit shown in FIG. 2.

FIG. 12 is a perspective view illustrating a still further example.

### DETAILED DESCRIPTION OF ADVANTAGEOUS EXAMPLES OF THE INVENTION

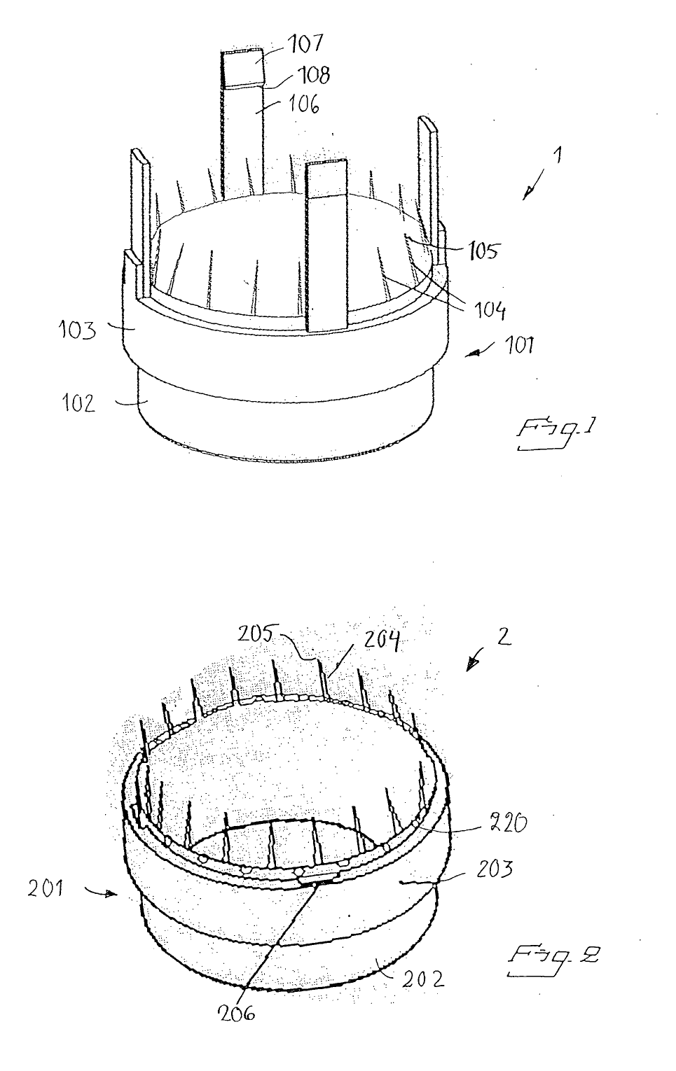

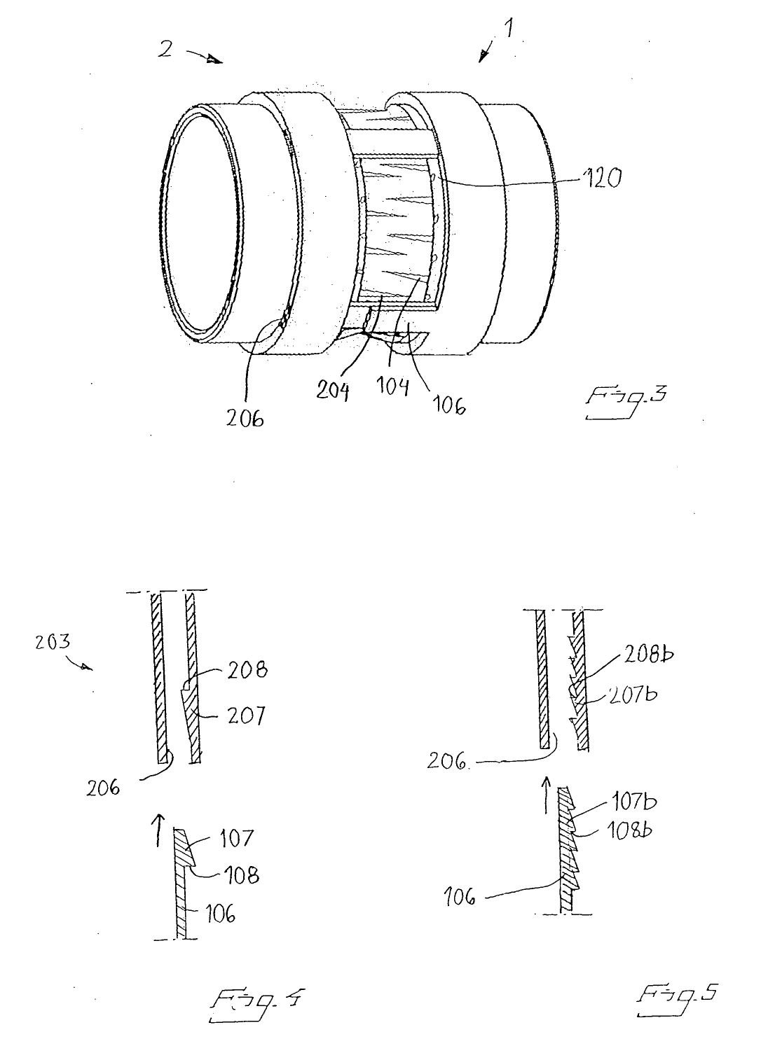

The anastomosis device consists of first 1 and second 2 attachment units, where FIG. 1 depicts the first unit 1, FIG. 2 the second unit 2 and FIG. 3 the two units connected to each other.

FIG. 1 thus is a perspective view of the first attachment unit 1. The unit has a body 101 that is generally cylindric. The body has a rear part 102 and a front part 103, the front part 103 having a slightly larger outer diameter than the rear part 102.

Means for attaching the units to a vessel end and means for connecting the unit to the second unit extend substantially axially from the front edge of the front part 103.

The means for attaching the unit to a blood vessel consists of a plurality of projections 104 with sharp edges 105. In the illustrated example these projections 104 are needle-like. The number of such projections can vary depending on the size of the vessel to which the unit is to be attached. Typically the number of projections 104 is in the range from 4 to 20.

The direction of each the projections 104 can preferably deviate slightly from the axial direction so that it has a small radial component in its extension, i.e. being slightly outwards inclined towards their edges.

The means for connecting the unit 1 to the second unit 2 consists of four axially extending members 106, in the illustrated example shaped as tongues. Also the number of tongues can vary but in most cases 4 tongues is appropriate. Each tongue 106 is flexible in the radial direction for allowing the connection with the second unit 2 as will be described later. Each tongue 106 at its outer end is provided with a radially offset part 107 creating a shoulder 108 where it joins the inner part of the tongue 106.

FIG. 2 is a perspective view of the second attachment unit 2. Also this unit has a body 201 that is generally cylindric and has a rear part 202 and a front part 203, where the front part 203 has slightly larger outer diameter than the rear part 202. The diameter of the body 201 corresponds to the diameter of the body of the first part 1.

Like the first unit 1, the second unit 2 is provided with a plurality of sharp needle-like projections 204 with edges 205.

At the edge of the front part 203 a number of axially directed openings 206 are provided. The number of the openings 206 corresponds to the number of tongues 106 of the first unit 1, and the cross sectional shape of each opening 206 generally corresponds to the cross sectional shape of each tongue 106.

At the edge of the front part 203 of the body 201 there are also provided a number of holes 220, corresponding to the number of projections 105 of the first unit 1. The holes 220 are arranged to receive the edges 105 of the projections 104 of the first unit 1.

The units 1 and 2 can be connected to each other by means of the tongues 106 and openings 206. FIG. 3 illustrates the units 1, 2 connected to each other. The connection is established by moving the units 1, 2 axially towards each other while the ends of the tongues 106 are inserted in the openings 206. Due to the flexibility of the tongues 106 in the radial direction they can snap into a locking position in the openings 206 so that the units are axially locked to each other.

In FIG. 3 it can be seen that also the first unit 1 is provided with holes 120 similar to the holes 220 of the second unit 2 as illustrated in FIG. 2.

The attachment units 1, 2 are preferably made of titanium. The attachment units can also be made of a resorbable material, partly or completely.

FIG. 4 is a schematic section through the end of one tongue 106 and one opening 206 and illustrates the snap-action and locking. As earlier described the end of tongue 106 has a radially offset part 107 forming a shoulder 108. One wall of the opening 206 in the second unit 2 is provided with a corresponding radially offset part 207 forming a shoulder 208.

When the end of the tongue 106 is inserted to the opening 206 as indicated by the arrow in the figure, the radially offset parts 107, 207 forces the end of the tongue 106 to be flexed to the left in the figure. When the shoulder 108 of the tongue 106 has passed the shoulder 208 of the opening 206 the end of tongue will flex back again and snap into a locking position where the shoulders 108, 208 abut against each other and lock the units against axial separating movement.

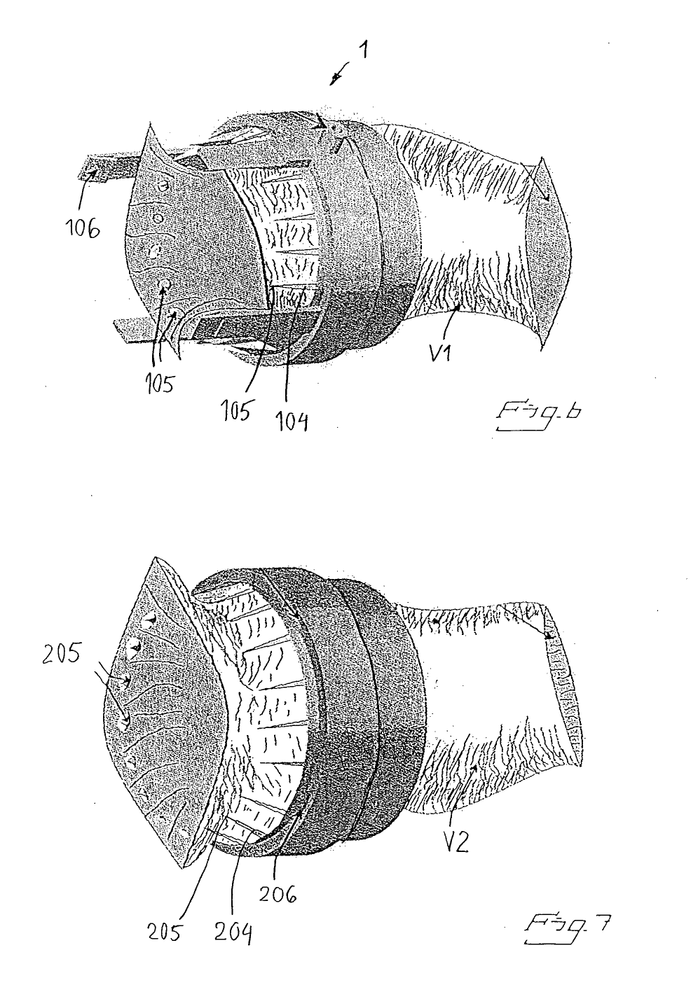

FIG. 5 illustrates in a corresponding section an alternative shape of the tongue 106 and the opening 206. In this case the tongue 106 at its outer end is serrated and thus has a saw-tooth profile 107b for cooperation with a corresponding saw-tooth-profile 207b on one wall of the opening. Thereby a plurality of shoulders 108b, 208b are formed that can cooperate with each other to obtain a locking position in various axial position. Thereby the tightness of the connection can be selected by the surgeon. Alternatively only either the tongue or the opening wall can be serated and the other having solely one shoulder.

FIG. 6 in a perspective view illustrates how the first attachment unit 1 is attached to the end of a vessel V1. First the end of the vessel V1 is led through the through-hole of the unit 1 from the rear end towards the front end until the end of the vessel V1 has passed the edges 105 of the needle-like projections 104 and a short distance further. Thereafter the end of the vessel V1 is folded outwards and back on the outside of the projections 104. As the end of the vessel V1 is drawn rearwards the edges 105 of the projections 104 penetrate the vessel wall whereby a secure attachment of the unit 1 to the vessel is established. For best result of the anastomosis the vessel intimae (internal layer) are in intimate contact. The surgeon should pay attention that all vessel layers are perforated by the edges 105 of the projections 104.

FIG. 7 in a corresponding view illustrates the attachment of the second attachment unit 2 to the end of a second vessel V2. This is accomplished in a similar way as explained in connection with FIG. 6 above.

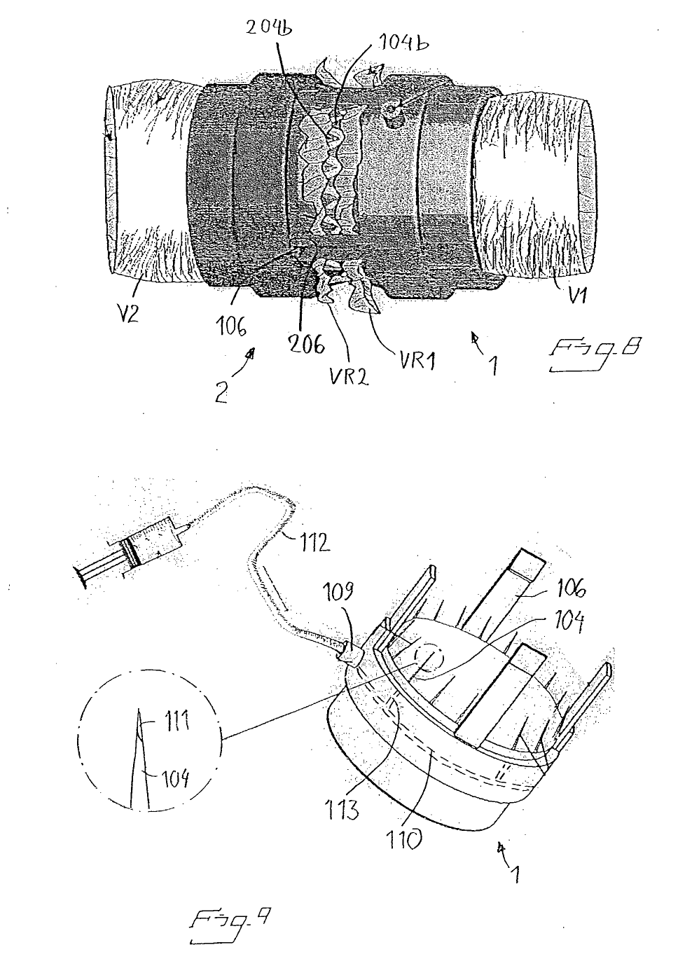

FIG. 8 in a side view illustrates how the two vessels V1, V2 are connected to each other after performing the attachment steps described in connection with FIGS. 6 and 7 and after connecting the two units 1, 2 as described in connection with FIG. 3.

The two vessels V1, V2 can be either of the same diameter or be of different diameter. By the way the vessel ends are reverted over the sharp edges of the projections a secure anchorage of the vessels is obtained and offers intima-to-intima connection of the two vessel ends.

In FIG. 8 a modified example of the shape of the sharp projection 104b, 204b is illustrated. In this example they have a general triangular shape instead of the needle-like shape earlier described. The reversed vessel walls that are folded back over the projections 104b, 204b are visible in the figure as VR1 and VR2.

FIG. 9 illustrates a further modified example of the first attachment unit 1. In this case the wall of the front end 103 is provided with a radial supply opening 109 communicating with a circumferentially extending channel 110 in the wall.

The channel 110 communicates via a channel 113 with the interior of the projection 104, which thus is hollow. At the outer end of the projection thus is an opening 111.

The radial supply opening 109 has a connection for connecting the opening to a conduit 112. A drug for preventing postoperative thrombosis, e.g. heparin thereby can be administrated directly to the anastomosis region and distributed via the supply opening 109 through the outlet openings 111 at the edge of the projection 104. Preferably the supply opening 109 is provided with a valve assuring one-way-flow. A one-way valve can alternatively be provided in each of the axial projections 104 that are provided with an outlet opening 111. The supply opening 109 is arranged in a small radial projection which preferably is made of biological slow resorbable material. Preferably at least four of the projections 104 are provided with such outlet openings. These projections 104 are inclined radially inwards.

Alternatively such a supply opening of course can be arranges on the second attachment unit 2.

As an alternative to a one-way-flow valve in the supply opening 109, a plug of silicon can be arranged in the supply opening 109. When supplying a drug this plug can be penetrated by a needle. After supplying the drug when the needle is withdrawn the hole made by the needle will automatically be closed.

The arrangement with a supply opening 109 can be utilized also with other types of attachment units than those illustrated and described in this application, e.g. having other kind of attachment means and other kind of connection means. The principle of supplying a drug through an opening can also be applied for end-to-side anastomosis and side-to-side anastomosis.

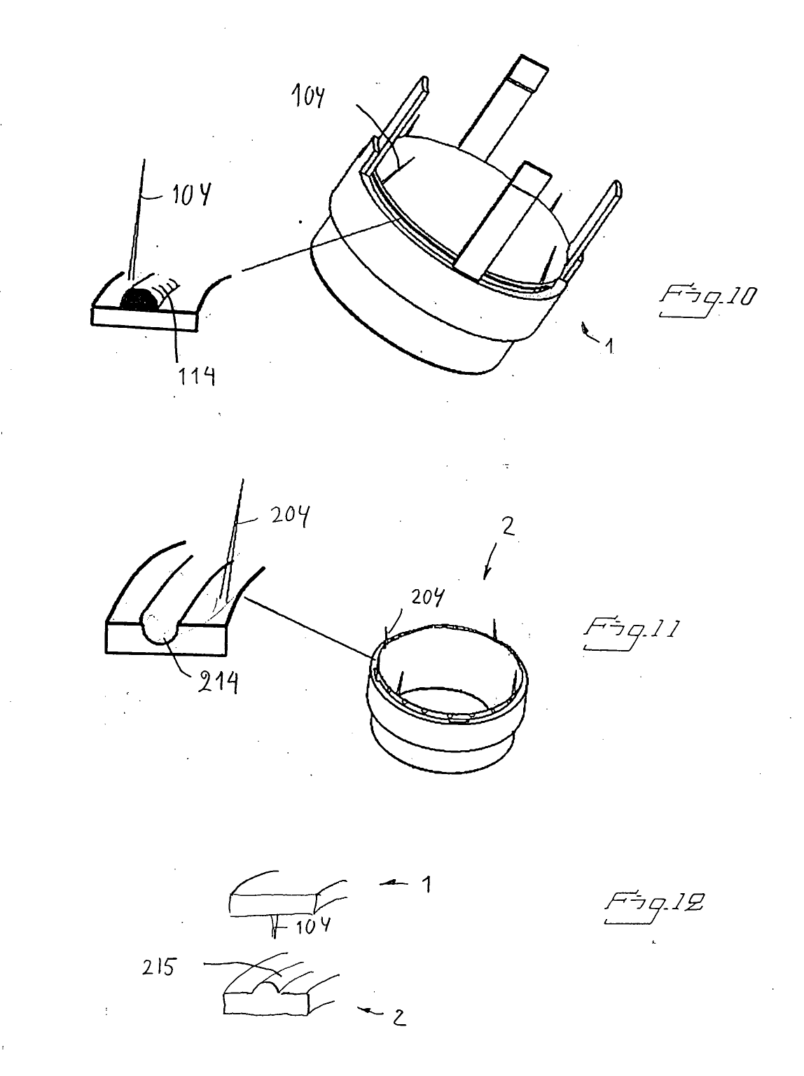

The attachment unit 1 in FIG. 10 is provided with a rim 114 on its end surface and attachment unit 2 in FIG. 11 is provided with a groove 214 on its end surface. The rim 114 and groove 214 have complementing shape such that when the units are connected to each other the rim 114 fits into the groove 214 and provides a tight seal.

In FIG. 12 the attachment unit 2 has a rim 215 on its end surface. This rim is located such that the needle 104 of unit 1 will penetrate into it when the units are connected. The rim is made of silicone. Correspondingly but not shown in the figure also the unit 1 can have such a rim to receive the needles (not shown) of the unit 2.

For manipulating the two units to be connected it is generally advantageous to use stapler.

1. An anastomosis device for anastomosing the ends of two vessel parts or other organs together, the device including first and second attachment units attachable to a respective end of the vessel parts and connectable to each other, each attachment unit having a through-hole defining a central axis of the attachment unit
wherein
the device is provided with at least two connection means, each connection means including an axially extending member having a free end, which member is provided on any of the attachment units and a receiving opening provided on the other of the attachment units, which receiving opening is arranged for axially receiving the free end of said member and which member is arranged to be able to snap into a locking position in said receiving opening in which position the member is axially locked.

2. A device according to claim 1 wherein all of said members are provided on the first attachment unit and all of said receiving openings are provided on the second attachment unit.

3. A device according to claim 2 wherein each member has such a shape that it is flexible to or from the central axis of the through-hole of the first attachment unit.

4. A device according to claim 3 wherein each member has a tongue-like shape having its thinnest dimension in the radial direction.

5. A device according to claim 3 wherein the free end of each member is provided with at least one tangentially extending shoulder, and one wall of each receiving opening is provided with at least one tangentially extending shoulder.

6. A device according to claim 5 wherein there is a plurality of axially distributed shoulders on each member and/or in each receiving opening such that a saw-tooth-like profile is formed on each member and in each receiving opening.

7. A device according to claim 1 wherein each attachment unit includes penetration means arranged to penetrate the walls of the related vessel thereby establishing attachment of the unit to the end of the vessel.

8. A device according to claim 7 wherein the penetration means includes a plurality of axially directed projections each projection having a sharp axially directed edge.

9. A device according to claim 8 wherein each attachment unit is provided with a plurality of holes arranged to receive the edges of the axially directed projections of the other unit.

10. A device according to claim 10 wherein each attachment unit is provided with a circumferentially extending rim of silicon arranged to receive the edges of the axially directed projections of the other unit.

11. A device according to claim 8 wherein each projection has a substantially triangular shape and has an acute angle forming said sharp edge.

12. A device according to claim 8 wherein each projection is shaped as a needle.

13. A device according to claim 8 wherein at least one of the axially directed projection are slightly radially inclined outwards in direction towards its edge.

14. A device according to claim 8 wherein the projections are circumferentially distributed around the through-hole.

15. A device according to claim 1 wherein at least one of said attachment units includes a supply opening and at least one outlet opening in communication with the supply opening, the supply opening being arranged to be connectable to a conduit, and each outlet opening being located such that it faces the interior of the vessel when the attachment unit is attached to the vessel.

16. A device according to claim 1 wherein a main part of each attachment unit is a substantially rotational-symmetric body.

17. A device according to claim 1 wherein at least one of said attachment units includes a supply opening and at least one outlet opening in communication with the supply opening, the supply opening being arranged to be connectable to a conduit, and each outlet opening being located such that it faces the interior of the vessel when the attachment unit is attached to the vessel.

18. A device according to claim 17 wherein at least one of said axially directed projections includes said at least one outlet opening located at or adjacent to the sharp edge of the projection.

19. A device according to claim 18 wherein each axially directed projection that includes one of said at least one outlet opening is slightly inclined radially inwards in direction towards its edge.

20. An anastomosis device for anastomosing two vessel parts together, the device including an attachment unit being attachable to a vessel part and being connectable to another attachment unit attached to another vessel part said attachment unit includes a supply opening and at least one outlet opening in communication with the supply opening, the supply opening being arranged to be connectable to a conduit, and the outlet opening being located such that it faces the interior of the vessel when the attachment unit is attached to the vessel.

21. A method for anastomosing of ends of two vessel parts together by attaching first and second attachment units to a respective end of the vessel parts and connecting the attachment units to each other, each attachment unit having a through-hole defining a central axis of the attachment unit
wherein the attachment units are connected to each other by at least two connection means, each connection means including an axially extending member provided on any of the attachment units and a receiving opening provided on the other of the attachment units, whereby the member is axially inserted into said receiving opening and snaps into the receiving opening such that the member becomes axially locked in the receiving opening.

22. A method according to claim 21 wherein the method is performed by using a device for anastomosing the ends of two vessel parts or other organs together, the device including first and second attachment units attachable to a respective end of the vessel parts and connectable to each other, each attachment unit having a through-hole defining a central axis of the attachment unit
wherein
the device is provided with at least two connection means, each connection means including an axially extending member having a free end, which member is provided on any of the attachment units and a receiving opening provided on the other of the attachment units, which receiving opening is arranged for axially receiving the free end of said member and which member is arranged to be able to snap into a locking position in said receiving opening in which position the member is axially locked.

23. A method for anastomosing two vessel parts together by attaching first and second attachment units to a respective vessel part and connecting the attachment units to each other, wherein a supply opening and at least one outlet opening are provided in one of the attachment units, a conduit is connected to said supply opening and a pharmacologically active liquid is supplied through the conduit, the supply opening and the outlet opening to the interior of the vessel to which the attachment unit is attached.

24. The use of a device according to claim 1 for anastomosing two vessels to each other.

25. The use according to claim 22 where said vessels are blood vessels.
